(19) United States
(12) Patent Application Publication
(10) Pub. No.: US 2020/0246519 A1
(43) Pub. Date: Aug. 6, 2020

(54) HANDS-FREE MILK EXTRACTION AND COLLECTION DEVICE

(71) Applicant: Amanda Quarles, Homestead, FL (US)

(72) Inventor: Amanda Quarles, Homestead, FL (US)

(21) Appl. No.: 16/268,496

(22) Filed: Feb. 6, 2019

(51) Int. Cl. A61M 1/06

(52) U.S. Cl. CPC A61M 1/064

(57) ABSTRACT

A hands-free milk extraction and collection device including a milk collection container and a housing unit. The milk collection container includes an internal cavity having a first section for receiving a nipple region of a woman's breast and having a second section for collecting milk extracted from the woman's breast. The housing unit is configured to latch onto the nipple region of the woman's breast to aid in stimulating milk flow from the breast by creating suction pressure at the nipple region. The housing unit having a shape contoured to a woman's breast and including a center opening for enclosing the milk collection container, wherein the housing unit and the enclosed milk collection container are collectively configured to fit inside a woman's bra for hands-free usage.

### FIELD OF THE INVENTION

Embodiments described herein generally relate to a milk collection device, and more particularly to a hands-free milk extraction and collection device.

### BACKGROUND OF THE INVENTION

Many women utilize a breast pump to induce milk lactation, which is stored and later bottle fed to their children. Although breast pumps allow mothers to conveniently prepare nursing bottles before feeding hours and/or serve to help mothers alleviate full breasts, most machines are handheld and too large, making milk collection a time-consuming task that may also make some individuals feel uncomfortable while doing so while on-the-go. Hence, it is desirable to provide an improved milk collection device.

### BRIEF DESCRIPTION OF THE DRAWINGS

The various advantages of the embodiments of the present disclosure will become apparent to one skilled in the art by reading the following specification and appended claims, and by referencing the following drawing(s), in which:

FIG. 1 shows an exemplary view of a hands-free milk extraction and collection device according to an embodiment of the present disclosure.

FIG. 2 shows another exemplary view of the hands-free milk extraction and collection device according to an embodiment of the present disclosure.

### SUMMARY OF THE INVENTION

Exemplary embodiments disclosed herein describe a hands-free milk extraction and collection device including a milk collection container and a housing unit. The milk collection container includes an internal cavity having a first section for receiving a nipple region of a woman's breast and having a second section for collecting milk extracted from the woman's breast. The housing unit is configured to latch onto the nipple region of the woman's breast to aid in stimulating milk flow from the breast by creating suction pressure at the nipple region. The housing unit having a shape contoured to a woman's breast and including a center opening for enclosing the milk collection container, wherein the housing unit and the enclosed milk collection container are collectively configured to fit inside a woman's bra for hands-free usage.

In some exemplary embodiments, the suction pressure at the nipple region is created without the use of an electric pump and without any manual pumping.

In some exemplary embodiments, the latching onto the nipple region of the woman's breast creates a seal around the nipple region.

In some exemplary embodiments, the housing unit latches onto the nipple region in a close-fitting manner.

In some exemplary embodiments, the suction pressure is created by the latching which effectuates squeezing of the nipple region of the breast, which causes extraction of milk from the breast.

In some exemplary embodiments, the device includes a plastic cap which is configured to fit into the woman's bra to keep the pressure from the bra from releasing the suction.

In some exemplary embodiments, the housing shell is a plastic shell.

In some exemplary embodiments, the milk collection container is plastic.

### DETAILED DESCRIPTION

The present disclosure relates to a hands-free milk extraction and collection device. The device fits into a nursing mother's bra for support. The device includes a suction mechanism that latches onto the nipple, which helps stimulate milk flow when milk let down occurs on the opposite breast (i.e., the non-nursing breast) while nursing. The milk will be collected in a milk collection container and can be poured for storage when the mother is done breastfeeding. The suction enables the device to not require electronic or manual pumping as the milk let down is a natural component of breastfeeding.

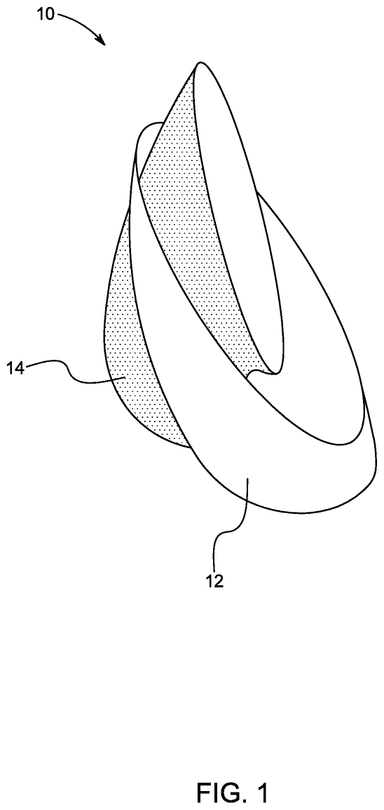

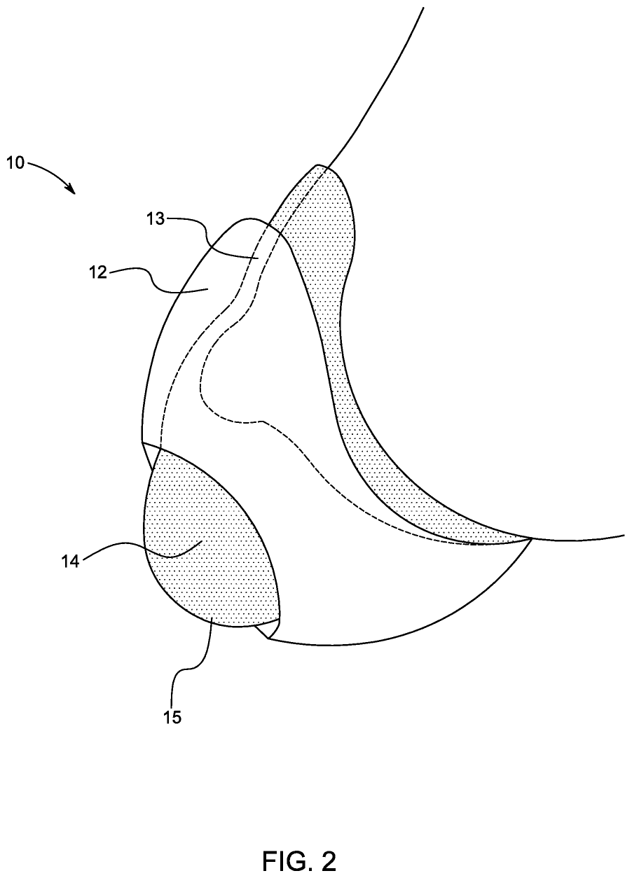

As illustrated in FIGS. 1-2, the hands-free milk extraction and collection device 10 (“the device”) includes a housing unit 12 and a milk collection container 14. The housing unit 12 has a shape contoured to a woman's breast and includes a center opening for enclosing the milk collection container 14. The housing unit is configured to latch onto the nipple region of the woman's breast to aid in stimulating milk flow from the breast by creating suction pressure at the nipple region.

The suction created by the latching of the housing unit 12 onto the nipple region of the breast is effected without the use of an electric pump and without any manual pumping. When the housing unit 12 latches onto the nipple region of the breast, a seal is created between the outer perimeter of the device 10 and the woman's breast around the nipple region. The housing unit latches onto the nipple region in a close-fitting (i.e., tight) manner which creates suction pressure by causing a squeezing and massaging effect to the nipple region. This suction pressure draws out (i.e., extracts) the milk directly from the breast and into the milk collection container 14.

The housing unit 12 may be made from any suitable material. In a preferred embodiment, the housing unit is made from plastic. The housing unit and the enclosed milk collection container are collectively configured to fit inside a woman's bra for hands free usage. In another exemplary embodiment, the device 10 includes a plastic cap which is configured to fit inside the woman's bra to keep the pressure from the bra from releasing the suction.

The milk collection container 14 includes an internal cavity having a first section 13 for receiving the nipple region of the woman's breast and having a second section 15 for collecting the milk extracted from the woman's breast. The milk collection container may be sized to collect any suitable amount of milk. In a preferred embodiment, the milk collection container is able to store six to eight ounces of milk. The milk collection container may be made from any suitable material, such as, for example, plastic or silicone.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.)

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

Those skilled in the art will appreciate from the foregoing description that the broad techniques of the embodiments of the present invention may be implemented in a variety of forms. Therefore, while the embodiments of this invention have been described in connection with particular examples thereof, the true scope of the embodiments of the invention should not be so limited since other modifications will become apparent to the skilled practitioner upon a study of the drawings, specification, and following claims.

What is claimed is:

1. A hands-free milk extraction and collection device comprising:
a milk collection container including an internal cavity having a first section for receiving a nipple region of a woman's breast and having a second section for collecting milk extracted from the woman's breast; and
a housing unit configured to latch onto the nipple region of the woman's breast to aid in stimulating milk flow from the breast by creating suction pressure at the nipple region, the housing unit having a shape contoured to a woman's breast and including a center opening for enclosing the milk collection container, wherein the housing unit and the enclosed milk collection container are collectively configured to fit inside a woman's bra for hands free usage.

2. The device of claim 1, wherein the suction pressure at the nipple region is created without the use of an electric pump and without any manual pumping.

3. The device of claim 1, wherein the latching onto the nipple region of the breast creates a seal around the nipple region.

4. The device of claim 1, wherein the housing unit latches onto the nipple region in a close-fitting manner.

5. The device of claim 1, wherein the suction pressure is created by the latching which effectuates squeezing of the nipple region of the breast, which causes extraction of milk from the breast.

6. The device of claim 1, further comprising a plastic cap which is configured to fit inside the woman's bra to keep the pressure from the bra from releasing the suction.

7. The device of claim 1, wherein the housing unit is a plastic shell.

8. The device of claim 1, wherein the milk collection container is plastic.
